(19) United States
(12) Patent Application Publication
(10) Pub. No.: US 2008/0075677 A1
(43) Pub. Date: Mar. 27, 2008

(54) Antioxidant Complex and a Cosmetic and Pharmaceutical Composition Comprising Said Complex

(76) Inventors: Rodrigo Fuscelli Pytel, Cajamar-SP (BR); Tereza M. de Senne Peranovich Victorio, Sao Paulo-SP (BR); Luciana Villa Nova Silva, Sao Paulo-SP (BR); Jean Luc Gesztesi, Sao Paulo-SP (BR); Edjane dos Santos Lima, Sao Paulo-SP (BR); Flavia da Silva Sena, Cajamar-SP (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/575,573

(22) PCT Filed: Sep. 2, 2005
(86) PCT No.: PCT/BR05/00200

§ 371 Date: Jul. 25, 2007

(30) Foreign Application Priority Data
Sep. 24, 2004 (BR) PI0404331-6

(51) Int. Cl. A61K 31/355; A61K 8/06; A61K 8/67; A61P 17/18; A61Q 17/00

(52) U.S. Cl. 424/59; 514/458

(57) ABSTRACT

The present invention relates to an antioxidant complex comprising from 0.000001% to 40.00000% by weight of at lest one cinnamiA acid, from 0.000001% to 40.00000% by weight of at least one carotenoid, from 0.000001% to 99.999998% by weight of vitamin E, and a cosmetically and pharmaceutically acceptable a carrier, all the amounts being based on the total weight of the antioxidant complex. The present invention further relates to cosmetic and pharmaceutical compositions comprising said antioxidant complex. The antioxidant complex of the present invention, as well as the above-cited compositions, aid in protecting and preventing cellular and cutaneous damages resulting from the action of free radicals.

This application claims the priority of Brazilian patent case No. P10404331-6 filed on Sep. 24, 2004 which is hereby incorporated by reference.

### FIELD OF THE INVENTION

The present invention relates to an antioxidant complex to aid in the protection and prevention of cellular and cutaneous damages resulting from the action of free radicals.

The present invention further relates to a cosmetic and pharmaceutical composition comprising said antioxidant complex.

### DESCRIPTION OF THE PRIOR ART

Free radicals and reactions triggered by these radicals are involved in a variety of physiological and pathological processes, including aging. It is well known that such radicals are produced in aerobic cells by agents of the environment, as well as from normal metabolic processes, such as cell breathing. It is also known that aerobic organisms have efficient defense mechanisms against free radicals, characterized by a number of enzymes and antioxidant compounds, which maintains these oxidant agents in sub-low concentrations.

However, when there is an unbalance between the process of producing and the process of destroying these radicals reactive with oxygen, a condition of oxidative stress arises, in which the oxidative attack of the biomolecules is favored. The cause relation observed between this oxidative stress and several pathologies and degenerative processes in humans has aroused the interest for exploring molecules capable of interfering with the generation of free radicals or with the reactions triggered by them, acting as antioxidants.

Free radicals are formed in the aerobic eukaryotic cells by successive steps of monoelectronic reductions of oxygen, sequentially generating the anion radicals superoxide (o₂), hydrogen peroxide (H₂O₂) and hydroxyl radicals (HO), according to the scheme shown below.

0₂+e⁻→O₂₋

2H+0₂₋+e→H₂O₂.

H₂O₂+Fe⁺²→Fe⁺³+HO^{·}+HO⁻(Fenton Reaction)

Among the reactive species of oxygen, the most powerful oxidant is represented by the hydroxyl radicals (HO^{·}), which are capable of reacting rapidly with virtually all the biomolecules, such as lipids, proteins, carbohydrates and nucleic acids, leading to the development of cellular toxicity. Therefore, substances capable of reacting with the hydroxyl radicals or preventing the prolongation of their radical reaction chains constitute valuable elements in the antioxidant protection of the biologic systems. Such substances are generically called sequestrants of free radicals or electron donors.

The application of isolated antioxidant components promotes an actuation thereof, with maximal power, preferably in lipophilic biological media or preferably in hydrophilic biological media, according to their polarity. Thus, isolated substances act more adequately against specific types of reactive species.

The combinations of antioxidant substances, in general, are unstable and promote great physicochemical alterations in products that contain them.

The applicant indicates, hereinafter, the relevant documents of the prior art relating to the matter of the present invention.

Document BR 0101449 of the applicant itself presents an antioxidant composition formed by tocopheryl acetate, lycopene, OPC glycospheres of grape seeds, sunflower-seed extract.

Document U.S. Pat. No. 5,023,235 describes an antioxidant system that comprises at least one stabilized ascorbyl ester with at least one complexing agent and at least one thiol. In this case, it is necessary to use an ascorbyl ester and a complexing agent so that the composition will be stable. Otherwise, the composition will be unstable, undergoing an intense yellowing after one week on the shelf.

Document KR 2001002411 describes a cosmetic composition containing stabilized vitamin C, but of a type of herb extract, exhibiting antioxidant activity.

Document WO 98/44905 describes cosmetic and dermatological compositions with antioxidant action, which contain Lapsana extract.

Document U.S. Pat. No. 6,066,311 describes a method of producing caffeic acid on a large scale and a composition that exhibits UV-absorbing activity, in addition to antioxidant properties.

Document WO 02/34210 describes the use of the association of at least one carotenoid and vitamin C in a composition or for preparing a composition. This composition acts on the treatment of cutaneous symptoms of aging, exhibiting the activity and/or expression of collagenase and increasing the synthesis of collagen.

From the description of the present invention hereinafter, it can be concluded that no teaching of the prior art proposes advantages referring to financial expenditures, stability of the cosmetic composition and quite high antioxidant potential, resulting in a better protection of the skin against cellular and cutaneous damages, without the need to add vitamin C, which would cause degradation and the consequent instability of the composition of the antioxidant complex.

### SUMMARY OF THE INVENTION

An objective of the present invention is to provide an antioxidant complex comprising 0.000001% to 40.00000% by weight of at least one cinnamic acid, 0.000001% to 40.00000% by weight of at least one carotenoid, 0.000001% to 99.999998% by weigh of vitamin E, and a cosmetic and pharmaceutically acceptable carrier, all the amounts being based on the total weight of the antioxidant complex.

Another objective of the present invention is to provide a cosmetic composition and a pharmaceutical composition comprising the antioxidant complex mentioned above.

### BRIEF DESCRIPTION OF THE FIGURES

The present invention will now be described in greater detail with reference to the figures that illustrate the performance tests of the present invention.

FIG. 1 illustrates the visual result of the comparative test between the antioxidant complex of the present invention and the same complex containing vitamin C, showing the alteration in color of the latter;

FIG. 2 illustrates the visual result of the comparative test between the antioxidant complex of the present invention and the same complex containing vitamin C, showing separation of the phases of the latter;

FIG. 3 illustrates the visual result of the comparative test between the antioxidant complex of the present invention and the same complex containing vitamin C, showing separation of phases of the latter with a roughened appearance;

FIG. 4 illustrates the visual result of the comparative test between the antioxidant complex of the present invention and the same complex containing vitamin C, showing oxidation of the latter.

### DETAILED DESCRIPTION OF THE INVENTION

The present invention describes an antioxidant complex comprising at least one cinnamic acid, which may be obtained, for instance, from coffee extract, at least one carotenoid, preferably lycopene, and vitamin E, in addition to a cosmetic and pharmaceutically acceptable carrier. This complex aids in the protection and prevention of cellular and cutaneous damages resuIting from the action of free radicals with high efficacy.

Antioxidant complex is defined herein as a composition comprising at least 3 components that exhibit antioxidant potential and, in combination, exhibit an unexpected synergistic effect, achieving a very high antioxidant potential.

The present invention further describes cosmetic and pharmaceutical compositions of topical use for skin care when subjected to UV rays, formed by a combination of water-soluble and liposoluble antioxidant agents in the same complex that enables an adequate actuation in both biologic media, both hydrophilic and lipophilic.

Further, in the specified amounts, it is not necessary to add vitamin C. In this regard, the composition is more advantageous in financial terms, in addition to the fact that it does not have the inevitable drawbacks caused by the degradation of this component, when present in cosmetic and/or pharmaceutical compositions.

The main examples of products that can be prepared from the antioxidant complex of the present invention or from cosmetic and pharmaceutical compositions comprising said complex are:
body moisturizing milk
face moisturizing milk
body moisturizing lotion
face moisturizing lotion
gels
products for the scalp
sunscreens or sun blocks for adults and children, intended for concomitant use in the practice of sports or not
body or face moisturizers
products against body and face stains
firming products for body and face
self-tanning products
insect-repelling products
body or face moisturizers that brightens the skin
pharmaceutical preparations of topical application
body and face cosmetic preparations for children
localized-action cosmetic preparations, specific for the periocular region, contour of the lips, lips, anti-stains, against rings under the eyes, among others
anti-acne products
products for clearing the skin
pharmaceutical compositions for the treatment of specific dermatoses.

The antioxidant complex of the present invention, incorporated into cosmetic or pharmaceutical composition, presents a variety of advantages and desired characteristics in a cosmetic or pharmaceutical product for the skin, some of which are listed below.

1. it is stable for a period of at least two years;

2. it exhibits an adequate non-sticky non-oily texture during the application;

3. it spreads easily;

4. it presents ideal time relating to the absorption and drying of the cosmetic product on the skin;

5. it does not cause oiliness on the skin after application;

6. it has high capability of maintaining the moisturizing of the skin;

7. it does not present comedogenidity;

8. it does not present phototoxicity;

9. it does not present allergenicity;

10. it does not cause any kind of adverse reaction or injury, be it cutaneous or ocular, either in normal conditions of use or in conditions of forced diaphoresis;

11. it is compatible with a wide variety of actives;

12. it provides a degree of skin moisturizing significantly above average of emulsions of the same category;

13. it provides smoothness, softness to the skin and has good spreadability, besides an excellent degree of homogeneousness and stability;

14. since it does not cause irritation, it becomes more comfortable and enables the use several times a day;

15. it exhibits adequate chemical stability;

16. it provides more protection of the skin against the formation of free radicals resulting from ultraviolet radiation, which has in its spectrum wave-length ranges that recognizably promote the formation of these species. In this latter sense, it permits formation of sunscreens and post-sun products that protect the skin more effectively against the damages caused by exposure to sunlight;

17. It provides more protection of the skin against the formation of free radicals resulting from exposure to various polluting agents, among them cigarette smoke. In this regard, it enables the preparation of formulation of products that form a film that protects the skin against these agents;

18. It provides more protection against the unbalance between the endogenous formation and combat of free radicals resulting from the natural aging process of the skin. In this way, it enables the preparation of more effective anti-signs formulations;

19. The antioxidant activity of the complex of the present invention is kept at adequate levels during the validity time of the products that contain it;

20. The shelf time of this complex is longer when compared with that of other agents and antioxidant systems of the prior art and further it does not need to be stored under cooled conditions;

21. The products that comprise the antioxidant complex of the present invention present absence of organoleptic alterations, unlike some products of the prior art, such as, for example:

a. products that contain tocopherol (a powerful antioxidant) may undergo intense yellowing, mainly when they are associated to another antioxidant or to some pro-oxidant agent;

b. products that contain vitamin C or derivatives thereof generally present significant alterations of aspect, color and odor.

22. The antioxidant complex of the present invention may be incorporated into various galenic forms, such as water gels, alcoholic gels, water-in-oil, oil-in-water, water-in-silicone emulsions sold under the names of gel creams, creams, lotions, among other possibilities of galenic forms, with maintenance of the stability of these compositions;

23. The antioxidant complex, when combined with at least one sunscreen, aids in protecting the immune system and exerts a DNA protecting activity;

24. The antioxidant complex of the present invention may be added to formulations with photoprotectors that contain physical sunscreens;

25. The antioxidant complex of the present invention comprises a combination of water-soluble and liposoluble antioxidant agents. Thus, it enables an adequate actuation in both biological media, both hydrophilic and lipophilic media, unlike most products of this line of the prior art.

### Antioxidant Complex

The antioxidant complex of the present invention comprises at least one cinnamic acid, which may be obtained, for instance, from coffee extract, at least one carotenoid, preferably lycopene and vitamin E. In addition to these components, the antioxidant complex may further comprise optional antioxidant agents.

On the other hand, the cosmetic and pharmaceutical compositions that comprise the antioxidant complex of the present invention may further contain various components such as photoprotectors agents (sunscreens) and other agents that exhibit specific functions required for each composition necessary for each situation such as sequestering agents, thickening agents, pH adjusting agents, preservatives, wetting agents, emollients, dyes, oiliness adsorbing agents, actives like anti-aging agents, among others.

The components that may be used in the antioxidant complex of the present invention will now be described in greater detail.

### Cinnamic acid

Preferably, coffee-bean extract may be added to the present invention, which comprises cinnamic acid in the form of chlorogenic acids and caffeic acid. This coffee-bean extract may be obtained, for instance, from the teachings of the French document FR 2 734 479. By this process a coffeebean extract rich in acids, mainly caffeic acid is obtained.

It is known from the prior art that the composition of coffee is: 1.5 to 2.5% of caffeine, which is bonded to chlorogenic acid, 3 to 5% of tannin and 10 to 13% of fixed oil (fatty oils). Chlorogenic acid imparts to coffee properties that regulate the SNC functions. It prevents coronary diseases, and depressions caused by heart diseases. In addition, it facilitates the learning and memorizing process. By the cited French process, a coffee bean extract with maximized amounts of the desired acids is obtained.

Further, from the documents of the prior art cited before, it can be concluded that caffeic acid exhibits antioxidant properties. Thus, for the present invention, preference is given to this acid.

In the preferred embodiments of the present invention, at least one cinnamic acid is added, preferably a mixture of chlorogenic acids, more preferably a mixture of chlorogenic acids rich in caffeic acid, in an amount ranging from 0.000001% to 40.000000%, preferably from 0.00001% to 10.00000%, more preferably from 0.001% to 0.01% by weight, based on the total weight of the composition of the antioxidant complex of the present invention.

### Vitamin E

In cosmetic formulations, the main forms found are tocopherol and tocopheryl acetate.

The former is used with better performance as an antioxidant, but since it has high instability with respect to light and air, it is often replaced by the latter, which has better stability.

Vitamin E is further found in the form of other tocopherol derivatives, such as tocopherol linoleate, tocopherol nicotinate and tocopherol palmitate.

Vitamin E acts as an antioxidant (against free radicals), further has anti-inflammatory capability and reduces the number of cells damaged by UV radiation.

In the preferred embodiments, vitamin E is added, preferably a tocopherol derivative, more preferably tocopheryl acetate in an amount ranging from 0.000001% to 99.999998%, preferably from 0.0001% to 10.0000%, more preferably from 0.01% to 2.00% by weight, based on the total weight of the composition.

### Carotenoid

Carotenoids are lipophilic pigments found in photosynthetic complexes of plants and photosynthetic bacteria, fungi and algae. More than 600 different natural carotenoids are known, wherein α-β-carotene, lycopene, β-cryptoxanthin, lutein and zeaxanthin are the carotenoids of higher concentration in the human plasma. The predominant carotenoids in the human epithelial tissue are β-carotene and lycopene; but the latter has a great advantage with respect to β-carotene, since it is not consumed during the “capture” of singlet oxygen and is not precursor of vitamin A, and may be used in products that are exposed to light.

In the preferred embodiments, at least one carotenoid is added, preferably lycopene or beta-carotene, more preferably lycopene extracted from tomato in an amount ranging from 0.000001% to 40.000000%, preferably from 0.00001% to 1.00000%, more preferably from 0.001% to 0.010% by weight, based on the total weight of the antioxidant complex.

### Optional Antioxidant Agent

Further, other optional antioxidant agents may be added to the antioxidant complex.

A few examples of compounds with antioxidant properties that may be added to the compositions of the present invention are: sulfites, imidazoles, urocanic acid and derivatives thereof, glutathione, lipophilic substances such as butyl hydroxytoluene (BHT), butyl hydroxyanisole (BHA), sunflower-seed extract and derivatives thereof, and others.

### DETAILS OF THE PRESENT INVENTION

The present invention does not comprise vitamin C, since this component would greatly restrict the types of formulation into which the anti-oxidant complex could be incorporated. If present in the formulations, vitamin C would become unstable, it would lose its therapeutic properties and cause significant alterations of color (making the products nut-brown or black) and significant olfactive alterations (imparting an unpleasant metallic odor):
formulations that contain oxides should not comprise vitamin C either, since said oxides function as catalysts of the degradation of vitamin C.

The present invention has a broad application in compositions containing oxides like, for example, titanium dioxide (TiO₂).
Oil-in-water emulsions, in which vitamin C (ascorbic acid) is solubilized in the aqueous medium, may present problems due to the contact of said vitamin directly with oxygen. In the reaction with oxygen vitamin C degrades. The cosmetic and pharmaceutical compositions that comprise the antioxidant complex of the present invention may be in this form.
When cosmetic and/or pharmaceutical formulations contain high contents of free water, it is easier to convert the molecular form of vitamin C to the ionized (more unstable) form. In this regard, since the cosmetic and pharmaceutical compositions of the present invention have free-water contents, the addition of vitamin C is avoided.

Images of the two formulations composed by the same base are given below, wherein one has added:
only the antioxidant complex of the present invention;
antioxidant complex of the present invention, associated to vitamin C.

The photographs register observations effected after a week of stability of the product.

Thus the present invention presents compositions that do not have the drawbacks found in some formulations of the prior art that contain vitamin C. Therefore, they do not present alterations of odor or separation of phases, present adequate coloration and prolonged stability.

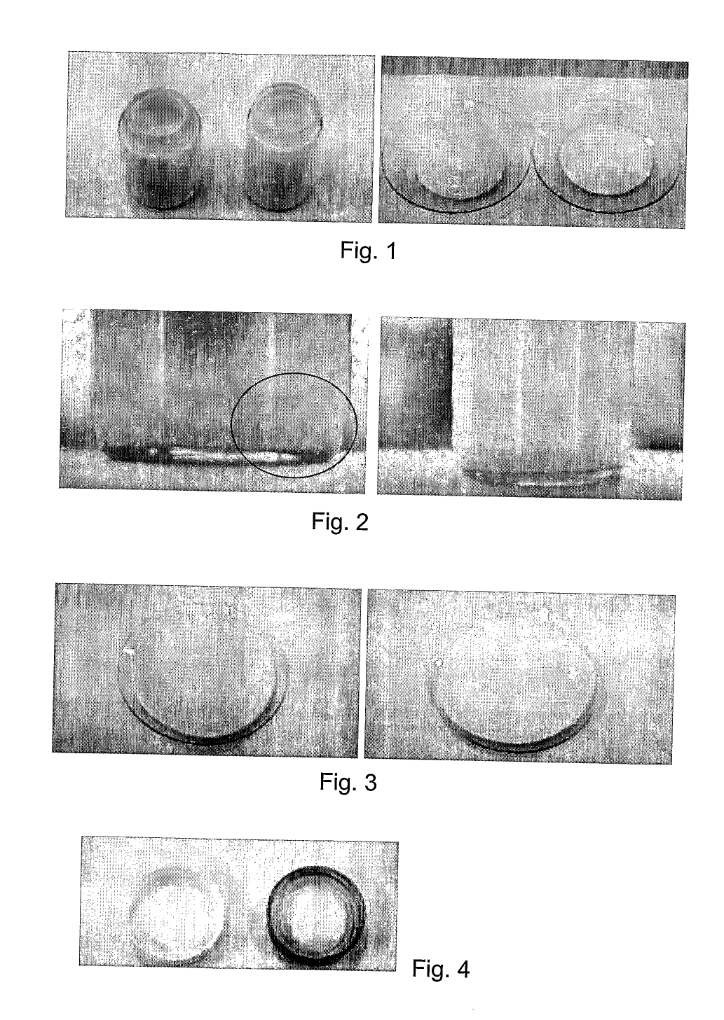

As can be seen in FIG. 1, the antioxidant complex, when comprising vitamin C, exhibits a significant alteration of color with respect to the sample, without the addition of vitamin C. On the other hand, the latter remains unchanged.

In FIG. 3, it can be noticed a significant alteration of color of the sample comprising vitamin C with respect to the sample without addition of vitamin C and further the former shows signs of separation of phases represented by clearer and dark spots on the bottom of the flask. The sample without addition of vitamin C remains unchanged.

Examining FIG. 3, it can be seen a significant alteration of color with respect to the sample comprising vitamin C with respect to the sample with addition of vitamin C and further the former also shows signs of separation of phases represented by the rough appearance present on the surface of the product. The sample without addition of vitamin C remains unchanged.

The sample comprising vitamin C of FIG. 4 still shows signs of irregular oxidation resulting from said vitamin, characterized by the more intense irregular alteration of color, where there more contact of the product with oxygen. The sample without addition of vitamin C remains unchanged.

All the compositions comprise the components of example 5 (only having, evidently, the difference of presence or absence of vitamin C) and were exposed to air in varying exposure times.

### Amounts Present in the Antioxidant Complex

By preference, the components of the antioxidant complex are present in the following proportions (all the amounts are expressed in parts by weight:
1^{st} composition:
   0.1 to 10 parts of a cinnamic acid;
   0.1 to 10 parts of a carotenoid;
   10 to 1000 parts of a tocopherol derivative.
2^{nd} composition:
   0.5 to 5 parts of a cinnamic acid;
   0.3 to 3 parts of a carotenoid;
   60 to 500 parts of a tocopherol derivative.
3^{rd} composition:
   0.8 to 1.5 part of a cinnamic acid;
   0.5 to 1.4 part of a carotenoid;
   90 to 200 parts of a tocopherol derivative.

According to the present invention, the antioxidant agents, when present in the proportions described herein, act with high efficacy, avoiding the need to add vitamin C, which is a widely known antioxidant agent. Thus, it prevents the drawbacks thereof already mentioned.

In addition, the carotenoid components, preferably lycopene, cinnamic acid, preferably chlorogenic acids rich in caffeic acid, vitamin E, preferably tocopheryl acetate, in the amounts specified herein achieve an antioxidant potential higher than expected. In comparison with antioxidant systems available on the market, said potential proves to be up to 7 times as high, thanks to the synergy obtained in combining said components in the defined amounts.

### Cosmetic and Pharmaceutical Compositions

The cosmetic composition, which may be an emulsion of the oil-in-water type, may comprise an amount of the antioxidant complex of the present invention ranging from 0.000003% to 70.00000% by weight, based on the total weight of said composition.

Further, the pharmaceutical composition, which may be an emulsion of the oil-in-water type, may comprise an amount of the antioxidant complex of the present invention ranging from 0.000003% to 99.999998% by weight, based on the total weight of said composition.

The cosmetic and pharmaceutical compositions that comprise the antioxidant complex of the present invention also may comprise some components known from the prior art.

### Emollients

The function of emollients in cosmetic compositions is to add or replace lipids and natural oils to the skin. They also act as solubilizers of sunscreens.

As emollients to be added to the composition of the present invention, one can cite conventional lipids like, for instance, oils, waxes, lipids and other water-insoluble components and polar lipids that are the lipids modified to increase their solubility in water by esterification of a lipid to a hydrophilic unit like, for example, hydroxyl groups, carbonyl groups, among others. Some compounds that may be used as emollients are natural oils such as essential oils and plant derivatives, esters, silicone oils, polyunsaturated fatty acids, lanoline and derivatives. Some natural oils that may be used are derived from damson seed oil, sesame oil, soybean, ground-nut, coco-nut, olive, cocoa-butter, almond, avocado, carnauba, rice bran, peach stone, mango stone, jojoba, macadamia nut, coffee bean, grape seed, pumpkin seed, among others and mixtures thereof.

Some ethers and esters may also be used in the function of emollients such as, for example, C₈-C₃₀ alkyl esters of C₈-C₃₀ carboxylic acids, C₁-C₆ diol monoesters and diesters of C₈-C₃₀ carboxylic acids, C₁₀-C₂₀ alcohol sucrose monoesters and combinations thereof. Examples of these compounds are dicaprylic ether, cetyl lactate, isopropyl palmitate, dicaprylyl carbonate, C₁₂₋₁₅ alkyl benzoate, isopropyl myristate, isopropyl isononate, sucrose palmitate, sucrose oleate, isostearyl lactate, glyceryl behenate, triglycerol-4 isostearate, lauryl pyrrolidone carboxylic acid, pantenyl triacetate and combinations thereof.

Silicones also act as emollients in the cosmetic and pharmaceutical compositions of the present invention. Some examples of silicones that may be added to said compositions are: volatile and non-volatile silicone oils such as, for example, cyclomethicone, alkyl dimethicones, dimethicone-copolyols, dimethiconols, phenyl trimethicones, capryllyl trimethicones, aminofuncional silicones, phenyl-modified silicones, alkyl-modified silicones; dimethyl and diethyl polysiloxane, mixed C₁-C₃₀ alkyl polysiloxane, polymethyl-siloxane, α-methyl-ω-methoxypolymethylsiloxane, polyoxydimethylsilylene, polydimethyl silicone oil and combinations thereof or silicone elastomers such as cyclomethicone dimethicone crospolymer, vinyl dimethicone and dimethicone crospolymer, dimethicone dimethicone crospolymer and cyclopenta-siloxane dimethicone crospolymer.

Other fatty alcohols, mono-, di- or triglyceride ethers that have a lipophilic nature as dicaprylylic ether may be used, in addition to synthetic and natural hydrocarbons, organic carbonates such as dicaprylyl carbonate, some types of silicones such as cyclomethicone and mixtures thereof.

In addition, several natural compounds may be used as emollient, as for example, microcrystalline wax, carnauba wax, Shea butter, beeswax, ozokerite wax, among others.

### Sunscreens

In order to filter the sunrays, sunscreen agents may be added, which may be water-soluble or liposoluble.

Some examples of sunscreens that absorb ultraviolet rays, which are indicated to be added to the cosmetic compositions of the present invention are: camphor benzylidene and derivatives thereof, camphor isophthalylidene and camphor terephthalylidene and derivatives thereof, cinnamic acid and esters thereof, salicylic acid and its esters, benzoic acid and its esters, p-aminobenzoic acid and derivatives thereof such as its esters, substituted hydrozylbenzophenones, substituted dibenzoylmethane, benzotriazole and some derivatives like 2-arylbenzotriazole, 2-arylbenzimidazole, 2-arylbenzofuranes, 2-arylbenzoxazole, 2-arylinol, monophenylcyanoacrylates, diphenyl-cyanoacrylates, among other ultraviolet-ray filters known from the prior art.

A few examples of sunscreens indicated for the present invention are organic compounds, often little soluble in water, such as triazine derivatives (for example, hydroxyphenyltrizine compounds or benzotriazole derivatives), some amides like those that contain a vinyl group, cinnamic acid derivatives, sulfonated benzimidazoles, oxalylamides, camphor derivatives, salicylic acid derivatives like 2-ethylhexyl, homoasalates and isopropyl salicylates, diphenylacrylates, benzophenone derivatives like benzophenone-2, benzophenone-3 and benzophenone-4, PABA like 2-ethylhexyl 4-dimethylamino-benzoate, and other sunscreens usually added to compositions of products for protection against sunlight.

In addition to the examples cited above, there are other preferred components for exerting this function, such as: anilino-N,N,N-trimethyl-4(2-oxoborn-3-ylidenomethy) methyl sulfate; 3,3′-(1,4-phenylenedimethylene) bis (7,7-dimethyl-2-oxo-bicyclo-(2.2.1) 1-hepthylmetanosulfonic acid and derivatives thereof; 1-(4 terc-butylphenyl)-3-(4-metoxyphenyl) propane-1,3-dione; α-(2-oxoborn-3-ylidene) toluene-4-sulfonic acid and its salts of potassium, sodium and trietanolamine; 2-cyano-3,2-ethylexyl 3′-diphenylacrylate; 2-etoxyethyl 4-metoxycinnamate; 2,2′-dihydroxy-4-metoxybenzophenone; methyl anthranylate; trietanolamine salicylate; 2, 2′, 4, 4′ tetrahydroxybenzophenone; 2-phenylbenzimidazol-5-sulfonic acid and its salts of potassium, sodium and trietanolamine; 2-ethylhexyl 4-methoxycinnamate; 2-hydroxy-4-metoxy-benzophenone (oxybenzone); 2-hydroxy-4-metoxybenzophenone 5-sulfonic acid and its sodium salt (sulisobenzone and sodic sulisobenzone); 4 amino-benzoic acid PABA; homomethyl salicylate; polymer of N-2{(2 and 4)[(2 oxo-born-3-ylidene) methyl] benzyl} acrylamide; titanium dioxide (with or without lipophilic coating); ethyl N-ethoxy-4-aminobenzoate; 2-ethylhexyl 4-dimethyla-minobenzoate; 2-ethylhexyl salicylate; isopentyl 4-ametoxycinnamate; 3-(4′-methylbenzilidene-no)-d-1-camphor; 3-benzilidene camphor; 2,4,6-trianinilin-(p-carbo-2′-ethyl-hexyl-1′-oxy)-1, e,5-triazine octyl; zinc oxide (with or without lipophilic coating); 2-(2H-benzotriazol-2-yl)-4-methyl-6-{2-methyl-3-(1,3,3,3-tetramethyl-1-((trimethylsilyl)oxy) disiloxanyl) propyl} phenol; benzoic acid; 4,4′[[6-[[40[[(1,1-dimethyl-ethyl) amino] carbonyl] phenyl] amino]-1,3k5-triazine-2,4-diyl] diimino] bis, bis (2 ethylhexyl); 2,2′-methylene-bis-6-(2H-benzo-triazol-2-yl)-4-(tetramethyl butyl)-1,1,3,3-phenol; methylene bis-benzotriazolyl tetraethyl butyl phenol; monosodic salt of 2,2′-bis-(1,4-phenylene)-1H-benzimidazol-4,6 disulfoni acid; (1,3,5)-triazine-2,4-bis {[4-(2-ethyl-hexyloxy)-2-hydroxyl]phenyl]-6-(4-metoxyphenyl); bis-ethylexyloxyphenol metoxyphenyl triazine; methylene bis-benzotriazolyl tetramethylbutylphenol; butyl metoxydi- benzoylmethane, p-metoxicinnamate of 2-ethylexyl, methylene bis-benzotriazolyl tetramethylbutylphenol, 4-butyl-4-metoxydibenziolmethane, benzophenone 3, bis-ethylhexyloxyphenol metoxyphenyl tirazine, octyl triazone, titanium dioxide, cinnamidopropyltrimonium chloride, dimiethylpabamidopropyl laurdimonium tosylate.

Further, there are other sunscreens that also protect the skin such as iron oxide, titanium dioxide, titanium dioxide combined with simethicone, zinc oxide, mono or polycarbonyl compounds like isatine, aloxane, ninidrine, glyceraldehyde, mesotartaric aldehyde, glutaraldehyde, pyrazolin-4,5-dione derivatives or 4,4-dihydroxypyrazolin-5-one derivatives, methylglyoxal, dialdehyde 2,3-dihydroxysuccinate, dialdehyde 2-amino-3-hydroxysuccinate and aldehyde 2-benzylamino-3-hydroxysuccinate.

### Carrier

Water is the base of several possibilities of cosmetic compositions prepared from the antioxidant complex already described, acting as a carrier for the other components. The compositions of the present invention comprise preferably demineralized or distilled water in a percentage (q.s.p.) suitable for achieving 100% of the formula, based on the total weight of the present composition. Naturally, other cosmetically acceptable carriers may be used in the present invention. In the examples of composition that will be described later, 96°0 GL ethyl alcohol is also used as a carrier.

### Other Optional Components

In order to impart to the cosmetic and pharmaceutical compositions of the present invention some desirable characteristic that is not achieved with the cited components, optional components that are compatible with it properties may be added. Some of these compounds that may be added to said compositions are the following:
active principles (either encapsulated or not): they may be either lipophilic or hydrophilic, such as seaweed extract, combination of palmitoyl hydroxypropyl trimonium aminopectin, glycerin crospolymer, lecithin and grape-seed extract, bisabolol (anti-inflammatory active), D-pantenol (conditioning active), biosaccharidic gum 2 and biosacharidic gum 3 and other actives usually added to compositions of products for topical use;
bacteriostatics, bactericides or antimicrobials;
emulsifier like potassium cetylphosphate, among others;
stabilizing agents such as sodium chloride, among others;
sequestering agents such as ethylenediaminetetraacetic acid (EDTA);
pH adjusting agent such as trietanolamine, sodium hydroxide, among others;
preservative like phenoxyethanol, PEG-5 cocoate and dicocoate of PEG-8 and iodopropinyl butylcarbamate and PEG-4, among others;
dyes of natural or synthetic origin;
thickening agents like xanthan gum and carbomer, among others;
plant extracts: chamomile, rosemary, thyme, calendula, carrot extract, common juniper extract, gentian extract, cucumber extract, among others;
skin conditioning agents; and
other cosmetically accepted components that are compatible with the antioxidant complex of the present invention.

### EXAMPLES OF COMPOSITION

The examples given below are preferred variations of compositions comprising the antioxidant complex of the present invention and should not be interpreted as being limitations thereof. In this regard, it should be understood that the scope of the present invention embraces other possible variations, being limited only by the contents of the accompanying claims, which include the possible equivalents.

### Example 1

### Emulsion without Sunscreen

| | | Mass |
|---|---|---|
| Component | Function | amount (%) |
| Water | Carrier | Qsp 100 |
| C12-15 alkyl benzoate | Emollient | 5.00 |
| Glycerin | Wetting agent | 5.00 |
| Dicaprylyl carbonate | Emollient | 2.00 |
| Potassium cethylfosfate | Emulsifying agent | 2.00 |
| Cyclomethicone | Emollient | 2.00 |
| Dimethicone and | Emollient | 1.50 |
| trimethylsiloxysilicate | | |
| Dimethicone stearyl | Emollient | 1.00 |
| Cyclomethicone and dimethicone | Emollient | 1.00 |
| copolyol | | |
| Phenoxyethanol | Preservative | 1.00 |
| Copolyol dimethicone methyl ether | Emollient | 0.50 |
| Vitamin E acetate | Antioxidant | 0.20 |
| PEG-5 Cocoate and PEG-8 dicocoate | Preservative | 0.20 |
| and iodopropinyl butylcarbamate and | | |
| PET-4 | | |
| Disodium EDTA | Sequestering agent | 0.10 |
| BHT | Antioxidant | 0.05 |
| Caffee extract (Coffea robusta) | Antioxidant | 0.002 |
| Lycopene extracted from tomato | Antioxidant | 0.002 |
| Xanthan gum | Thickening agent | 0.200.20 |
| Carbomer | Thickening agent | 0.35 |
| Triethanolamine | pH adjuster | 0.50 |

### Example 2

### Emulsion for Children SPF 30 (Sun Protection Factor)

| | | Mass |
|---|---|---|
| Component | Function | amount (%) |
| Water | Carrier | Qsp 100 |
| 2-ethylhexyl p-methoxycennamate | Sunscreen | 7.50 |
| C12-15 alkyl benzoate | Emollient | 5.00 |
| Glycerin | Wetting agent | 5.00 |
| Benzofenone 3 | Sunscreen | 5.00 |
| Dicaprylyl carbonate | Emollient | 3.50 |
| Potassium cethylphosphate | Emulsifying | 2.00 |
| | agent | |
| Cyclomethicone | Emollient | 2.00 |
| Titanium dioxide and simethicone | Sunscreen | 2.00 |
| Dimethicone and trimethylsiloxysilicate | Emollient | 1.50 |
| Bis-ethylhexyloxyphenol | Sunscreen | 1.50 |
| Stearyl dimethicone | Emollient | 1.00 |
| Cyclomethicone and dimethicone | Emollient | 1.00 |
| copolyol | | |
| Penoxyethanol | Preservative | 1.00 |
| Copolyol dimethicone methyl ether | Emollient | 0.50 |
| Vitamin E acetate | Antioxidant | 0.20 |
| PEG-5 cocoate and PEG-8 dicocoate | Preservative | 0.20 |
| and iodopropinyl butylcarbamate | | |
| and PET-4 | | |
| Disodium EDTA | Sequestering | 0.10 |
| | agent | |
| BHT | Antioxidant | 0.05 |
| Coffee extract (Coffea robusta) | Antioxidant | 0.002 |
| Lycopene extracted from tomato | Antioxidant | 0.002 |
| Xanthan gum | Thickening | 0.20 |
| | agent | |
| Carbomer | Thickening | 0.40 |
| | agent | |
| Trietanolamine | pH adjuster | 0.50 |
| Tartrazine yellow | Dye | 0.032 |
| FD&C REd N. 4 Ci 147000 | Dye | 0.003 |
| Liquid sorbitol | Solubilizer | 1.279 |
| Preservative GD 700 | Preservative | 0.023 |

### Example 3

### Moisturizing Protecting Emulsion SPF 30-01

| | | Mass |
|---|---|---|
| Component | Function | amount (%) |
| Water | Carrier | Qsp 100 |
| 2-ethylhexyl p-methoxycinnamate | Sunscreen | 7.50 |
| C12-15 alkyl benzoate | Emollient | 6.00 |
| Glycerin | Wetting agent | 5.00 |
| Benzophenone 3 | Sunscreen | 5.00 |
| Dicaprylyl carbamate | Emollient | 4.00 |
| Cyclomethicone | Emollient | 3.00 |
| Titanium dioxide and simethicone | Sunscreen | 3.00 |
| Dimethicone and trimethylsiloxysilicate | Emollient | 1.50 |
| Bis-ethylhexyloxyphenol methoxyphenyl | Sunscreen | 2.00 |
| triazine | | |
| Aluminum octenylsuccinate starch | Oil absorber | 1.50 |
| Stearyl dimethicone | Emollient | 1.00 |
| Potassium cetylphophate | Emulsifying | 2.00 |
| | agent | |
| Cyclomethicone and dimethicone copolyol | Emollient | 1.00 |
| Phenoxyethanol | Preservative | 1.00 |
| Vitamin E acetate | Antioxidant | 0.20 |
| PEG-5 cocoate and PEG-8 dicocoate and | Active | 0.20 |
| iodopropinyl sutylcarbamate and PEG-4 | | |
| Bisaccharide gum 2 | active | 0.20 |
| Bisaccharide gum 3 | Active | 0.20 |
| Disodium EDTA | Sequestering | 1.10 |
| | agent | |
| BHT | Antioxidant | 0.05 |
| Coffee extract (Coffea robusta) | Antioxidant | 0.002 |
| Lycopene extracted from tomato | Antioxidant | 0.002 |
| Xanthan gum | Thickening | 0.20 |
| | agent | |
| Carbomer | Thickening | 0.40 |
| | agent | |
| Trietanolamine | pH adjuster | 0.50 |

### Example 4

### Moisturizing Protecting Emulsion SPF 30-02

| | | Mass |
|---|---|---|
| Component | Function | amount (%) |
| Water | Carrier | Qsp 100 |
| 2-ethylhexyl p-methoxycinnamate | Sunscreen | 7.50 |
| C12-15 alkyl benzoate | Emollient | 6.00 |
| Glycerin | Wetting agent | 5.00 |
| Benzophenone 3 | Sunscreen | 5.00 |
| Dicaprylyl carbonate | Emollient | 3.50 |
| Cyclomethicone | Emollient | 3.00 |
| Titanium dioxide and simethicone | Sunscreen | 2.00 |
| Dimethicone and trimethylsiloxysilicate | Emollient | 1.50 |
| Bis-ethylhexyloxyphenol methoxyphenyl | Sunscreen | 1.50 |
| triazine | | |
| Stearyl dimethicone | Emollient | 1.00 |
| Potassium cetylphosphate | Emulsifying | 2.00 |
| | agent | |
| Cyclomethicone and dimethicone copolyol | Emollient | 1.00 |
| Phenoxyethanol | Preservative | 1.00 |
| Copolyol dimethicone methyl ether | Antioxidant | 0.50 |
| Vitamin E acetate | Antioxidant | 0.20 |
| PEG-5 cocoate and PEG-8 dicocoate and | Preservative | 0.20 |
| iodopropinyl butylcarbamate and PEG-4 | | |
| Bisaccharide gum 2 | active | 0.20 |
| Bisaccharide gum 3 | Active | 0.20 |
| Disodium EDTA | Sequestering | 0.10 |
| | agent | |
| BHT | Antioxidant | 0.05 |
| Coffee extract (Coffea robusta) | Antioxidant | 0.002 |
| Xanthan gum | Thickening | 0.20 |
| | agent | |
| Carbomer | Thickening | 0.40 |
| | agent | |
| Triethanolamine | pH adjuster | 0.50 |

### Example 5

### Protector SPF 30

| | | Mass |
|---|---|---|
| Component | Function | amount (%) |
| Water | Carrier | Qsp 100 |
| 2-ethylhexyl methoxycinnamate | Sunscreen | 7.50 |
| C12-15 alkyl benzoate | Emollient | 6.00 |
| Glycerin | Wetting agent | 5.00 |
| Benzophenone 3 | Sunscreen | 5.00 |
| Dicaprylyl carbonate | Emollient | 3.50 |
| Cyclomethicone | Emollient | 2.00 |
| Titanium dioxide and simethicone | Sunscreen | 2.00 |
| Dimethicone and trimethylsiloxysilicate | Emollient | 2.00 |
| Bis-ethylhexyloxyphenol methoxyphenyl | Sunscreen | 1.50 |
| triazine | | |
| Stearate dimethicone | Emollient | 1.00 |
| Potassium cetylphosphate | Emulsifying | 2.00 |
| | agent | |
| Cyclomethicone and dimethicone | Emollient | 1.00 |
| copolyoil | | |
| Phenoxyethanol | Preservative | 1.00 |
| Copolyol dimethicone methyl ether | Emollient | 1.00 |
| Vitamin E acetate | Antioxidant | 0.20 |
| PEG-5 Cocoate and PEG-8 dicocoate and | Preservative | 0.20 |
| iodopropinyl butylcarbamate and PEG-4 | | |
| Disodium EDTA | Sequestering | 0.10 |
| | agent | |
| BHT | Antioxidant | 0.05 |
| Coffee extract (Coffea robsuta) | Antioxidant | 0.002 |
| Lycopene extracted from tomato | Antioxidant | 0.002 |
| Xanthan gum | Thickening | 0.20 |
| | agent | |
| Carbomer | Thickening | 0.40 |
| | agent | |
| Triethanolamine | Ph adjuster | 0.50 |

### Example 6

### Moisturizing Protecting Dry Fluid SPF 15

| | | Mass |
|---|---|---|
| Component | Function | amount (%) |
| 96° GL ethyl alcohol | Carrier | Qsp 100 |
| Clycerin | Wetting agent | 2.00 |
| Lycopene extracted from tomato | Antioxidant | 0.002 |
| Tocopherol acetate | Antioxidant | 0.20 |
| 96° GL ethyl alcohol | Carrier | 0.50 |
| Coffee extract (Coffea robusta) | Antioxidant | 0.002 |
| PVP/VA E-735 copolymer | Film former | 1.00 |
| Bis-ethylhexylphenol methoxyphenyl | Sunscreen | 1.00 |
| triazine | | |
| Dicapryl carbonate | Emollient | 10.00 |
| Benzophenone 3 | Sunscreen | 4.00 |
| Octal triazone | Sunscreen | 3.00 |
| 2-ethyl hexyl p-methoxycinnamate | Sunscreen | 7.50 |
| BHT | Antioxidant | 0.05 |
| C12-15 alkyl benzoate | Emollient | 5.00 |
| Cyclomethicone | Emollient | 15.00 |

### Example 7

### Moisturizing Protecting Emulsion SPF 30-03

| | | Mass |
|---|---|---|
| Component | Function | amount (%) |
| Water | Carrier | Qsp 100 |
| 2-ethylhexyl p-methoxycinnamate | Sunscreen | 7.50 |
| C12-15 aliyl benzoate | Emollient | 6.00 |
| Clycerine | Wetting agent | 5.00 |
| Benzophenone 3 | Sunscreen | 5.00 |
| Dicaprylyl carbonate | Emollient | 3.50 |
| Cyclomethicone | Emollient | 2.00 |
| Titanium dioxide and simethicone | Sunscreen | 2.00 |
| Dimethicone and trimethyloxysilicate | Emollient | 1.50 |
| Bis-ethylhexyloxyphenol methoxyphenyl | Sunscreen | 1.50 |
| triazine | | |
| Dimethicone stearyl | Emollient | 1.00 |
| Potassium cetylphosphate | Emulsifying | 2.00 |
| | agent | |
| Cyclomethicone and dimethicone copolyol | Emollient | 1.00 |
| Phenoxyethanol | Preservative | 1.00 |
| Copolyol dimethicone methyl ether | Emollient | 0.50 |
| Vitamin E acetate | Antioxidant | 0.20 |
| PEG-5 cocoate and PEG-8 dicocoate and | Preservative | 0.20 |
| iodopropinyl butylcarbamate and PEG-4 | | |
| Disodium EDTA | Sequestering | 0.10 |
| | agent | |
| BHT | Antioxidant | 0.05 |
| Coffee extract (Coffea robusta) | Antioxidant | 0.002 |
| Lycopene extracted from tomato | Antioxidant | 0.002 |
| Xanthan gum | Thickening | 0.20 |
| | agent | |
| Carbomer | Thickening | 0.40 |
| | agent | |
| Trietanomaline | pH adjuster | 0.50 |

### Example 8

### Moisturizing Protecting Emulsion SPF 15

| | | Mass |
|---|---|---|
| Component | Function | amount (%) |
| Water | Carrier | Qsp 100 |
| 2-ethyhelyl p-methoxycinnamate | Sunscreen | 7.00 |
| C12-15 alkyl benzoate | Emollient | 5.00 |
| Clycerine | Wetting agent | 5.00 |
| Benzophenone 3 | Sunscreen | 1.00 |
| Dicaprylyl carbonate | Emollient | 2.00 |
| Cyclomethicone | Emollient | 2.00 |
| Titanium dioxide and simethicone | Sunscreen | 1.00 |
| Dimethicone and trimethylsiloxysilicate | Emollient | 1.50 |
| Bis-ethylhexyloxyphenol methoxyphenyl | Sunscreen | 1.00 |
| triazine | | |
| Stearyl dimethicone | Emollient | 1.00 |
| Potassium cetylphosphate | Emulsifying | 2.00 |
| | agent | |
| Cyclomethicone and dimethicone copolyol | Emollient | 1.00 |
| Phenoxyethanol | Preservative | 1.00 |
| Copolyiol dimethicone methyl ether | Emollient | 0.50 |
| Vitamin E acetate | Antioxidant | 0.20 |
| PEG-5 cocoate and PEG-8 dicocoate and | Preservative | 0.20 |
| iodopropinyl butylcarbanate and PGE-8 | | |
| Disodium EDTA | Sequestering | 0.10 |
| | agent | |
| BHT | Antioxidant | 0.05 |
| Coffee extract (Coffea robusta) | Antioxidant | 0.002 |
| Lycopene extracted from tomato | Antioxidant | 0.002 |
| Xanthan gum | Thickening | 0.20 |
| | agent | |
| Carbomer | Thickening | 0.35 |
| | agent | |
| Triethanolamine | pH adjuster | 0.50 |

### Example 9

### Moisturizing Protecting Emulsion SPF 8

| | | Mass |
|---|---|---|
| Component | Function | amount (%) |
| Water | Carrier | Qsp 100 |
| 2-ethyhelyl p-methoxycinnamate | Sunscreen | 4.50 |
| C12-15 alkyl benzoate | Emollient | 5.00 |
| Clycerin | Wetting agent | 5.00 |
| Benzophenone 3 | Sunscreen | 1.00 |
| Dicaprylyl carbonate | Emollient | 2.00 |
| Cyclomethicone | Emollient | 2.00 |
| Titanium dioxide and simethicone | Sunscreen | 1.00 |
| Dimethicone and trimethylsiloxysilicate | Emollient | 1.50 |
| Bis-ethylhexyloxyphenol methoxyphenyl | Sunscreen | 1.00 |
| triazine | | |
| Stearyl dimethicone | Emollient | 1.00 |
| Potassium cetylphosphate | Emulsifying | 2.00 |
| | agent | |
| Cyclomethicone and dimethicone copolyol | Emollient | 1.00 |
| Phenoxyethanol | Preservative | 1.00 |
| Copolyiol dimethicone methyl ether | Emollient | 0.50 |
| Vitamin E acetate | Antioxidant | 0.20 |
| PEG-5 cocoate and PEG-8 dicocoate and | Preservative | 0.20 |
| iodopropinyl butylcarbanate and PGE-8 | | |
| Disodium EDTA | Sequestering | 0.10 |
| | agent | |
| BHT | Antioxidant | 0.05 |
| Coffee extract (Coffea robusta) | Antioxidant | 0.002 |
| Lycopene extracted from tomato | Antioxidant | 0.002 |
| Xanthan gum | Thickening | 0.20 |
| | agent | |
| Carbomer | Thickening | 0.35 |
| | agent | |
| Triethanolamine | pH adjuster | 0.50 |

### Test

Objective: analyzing the antioxidant potential of the complex of the present invention in comparison with other known antioxidant system compositions.

A solution of 0.004% of DPPH (diphenyl pycryl hydrazyl) in ethanol was used. The action of DPPH on ethanol generates free radicals, forming colorful aducts, which are spectrophotometrically detected.

Further, a solution of BHT in ethanol was used and its solutions described in the table below.

| Component | | Product 1 | Product 2 | Product 3 |
|---|---|---|---|---|
| Tocopheryl acetate | | 5 | 3 | 3 |
| Lycopene | | 0.02 | 0.20 | 0.02 |
| OPC | | 2 | — | — |
| Helioxine | | 2.5 | — | — |
| Mixture of chlorogenic acids rich in | | — | 0.2 | 0.1 |
| caffeic acid | | | | |
| Alpha-totopherol | | — | — | — |
| Ascorbic acid | | — | — | — |
| Total % of actives | | 9.5 | 2.2 | 2.1 |
| Component | | Product 4 | Product 5 | Product 6 |
| Tocopheryl acetate | | 2 | 2 | 2 |
| Lycopene | | 0.02 | 0.02 | 0.02 |
| OPC | | — | — | — |
| Helioxine | | — | — | — |
| Mixture of chlorogenic acids reich in | | 0.067 | 0.04 | 0.04 |
| caffeic acid | | | | |
| Alpha-tocopherol | | — | — | 0.20 |
| Ascorbic acid | | — | — | — |
| Total % of actives | | 2.1 | 2.06 | 2.26 |
| Component | Product 7 | Product 8 | Product 9 | Product 10 |
| Helioxine | — | — | — | — |
| Mixture of chlorogenic | 0.02 | 0.05 | 0.05 | 0.02 |
| acids rich in caffeic acid | | | | |
| Alpha-tocopherol | — | 0.20 | 3.00 | — |
| Ascorbic acid | — | — | — | 0.02 |
| Total % of actives | 2I01 | 2.24 | 5.04 | 2.06 |

Tubes with the DPPH solution and the compositions of the above products were prepared.

According to the study, the anti-radical activity of the mixture of chlorogenic acids is directly proportional to the amount added to with respect to the volume of ethanol of the solution.

The antioxidant potential of the composition of the antioxidant complex comprising the mixture of chlorogenic acids rich in caffeic acid proved to be 7 times as high as the antioxidant potential of compositions that do not contain such a mixture. Therefore, an unexpected synergy is expected between the components present in the antioxidant complex of the present invention. Further, it has been noticed that the presence of vitamin C in this composition does not improve the potential of the antioxidant complex and, at determined concentrations, it causes reduction of said potential.

1. An antioxidant complex characterized by comprising from 0.000001% to 40.00000% by weight of at least one cinnamic acid, from 0.000001% to 40.00000% by weight of at least one carotenoid, from 0.000001% to 99.999998% by weight of vitamin E, and a cosmetically and pharmaceutically acceptable carrier, all the amounts being based on the total weight of the antioxidant complex.

2. An antioxidant complex according to claim 1, characterized in that the cinnamic acid is a mixture of chlorogenic acids.

3. An antioxidant complex according to claim 1, characterized in that the cinnamic acid comes from coffee extract.

4. An antioxidant complex according to claim 1, characterized by comprising an amount of cinnamic acid ranging from 0.00001% to 1.00000% by weight, based on the total weight of the complex.

5. An antioxidant complex according to claim 4, characterized by comprising an amount of cinnamic acid ranging from 0.001% to 0.01% by weight, based on the total weight of the complex.

6. An antioxidant complex according to claim 1, characterized by comprising an amount of carotenoid ranging from 0.00001% to 1.0000% by weight, based on the total weight of the complex.

7. An antioxidant complex according to claim 6, characterized by comprising an amount of carotenoid ranging from 0.001% to 0.010% by weight, based on the total weight of the complex.

8. An antioxidant complex according to claim 1, characterized in that the carotenoid is lycopene.

9. An antioxidant complex according to claim 1, characterized by comprising an amount of vitamin E ranging from 0.0001% to 10.0000% by weight, based on the total weight of the complex.

10. An antioxidant complex according to claim 9, characterized by comprising an amount of vitamin E ranging from 0.01% to 2.00% by weight, based on the total weight of the complex.

11. An antioxidant complex according to claim 1, characterized in that the vitamin E is tocopherol.

12. An antioxidant complex according to claim 1, characterized in that the vitamin E is tocopheryl acetate.

13. An antioxidant complex according to claim 1, characterized by comprising an additional antioxidant agent.

14. An antioxidant complex according to claim 13, characterized in that the additional antioxidant agent is BHT.

15. A cosmetic composition characterized by comprising, as an antioxidant agent, a complex as defined in claim 1.

16. A cosmetic composition according to claim 15, characterized by comprising an amount of antioxidant complex ranging from 0.000003% to 70.00000% by weight, based on the total weight of such a composition.

17. A cosmetic composition according to claim 16, characterized in that it is an emulsion of the oil-in-water type.

18. A cosmetic composition according to claim 16, characterized by comprising at least one sunscreen.

19. A pharmaceutical composition characterized by comprising, as an antioxidant agent, a complex as defined in claim 1.

20. A pharmac eutical composition according to claim 19, characterized by comprising an amount of antioxidant complex ranging from 0.000003% to 99.999998% by weight, based on the total weight of the composition.

21. A pharmaceutical composition according to claim 19, characterized in that it is an emulsion of the oil-in-water type.
